(19) United States
(12) Patent Application Publication
(10) Pub. No.: US 2009/0182148 A1
(43) Pub. Date: Jul. 16, 2009

(54) PROCESS FOR THE MANUFACTURE OF MONTELUKAST SODIUM

(76) Inventors: Harmander Singh Pal Chawla, Gujarat (IN); Anil Shankar Chowdhary, Gujarat (IN); Ajay Mangubhai Patel, Gujarat (IN); Vipul Narbheshankar Joshi, Gujarat (IN); Manish Popatial Patel, Gujarat (IN)

Correspondence Address: ARENT FOX LLP, 1050 CONNECTICUT AVENUE, N.W., SUITE 400, WASHINGTON, DC, 20036, US

(21) Appl. No.: 12/097,294

(22) PCT Filed: Jan. 16, 2006
(86) PCT No.: PCT/IB06/00059

§ 371 Date: Dec. 23, 2008

(30) Foreign Application Priority Data
Dec. 23, 2005 (IN) 1613/MUM/2005

(51) Int. Cl. C07D 215/14

(52) U.S. Cl. 546/174

(57) ABSTRACT

An improved process for the manufacture of 1-[[[(1R)-1-[3-[(1E)-2-(7-chloro-2-quinolinyl)ethenyl]phenyl]-3-[2-(1-hydroxy-1-methylethyl)phenyl]propyl]thio]methyl]cyclopropane acetic acid, sodium salt [montelukast sodium(I)]. It consists of converting a vital intermediate 1-(mercaptomethyl)-cyclopropane acetic acid of formula IX, to a quaternary ammonium salt of formula X, using a suitable base in a suitable solvent at 0° C. to 50° C. followed by monometalation to provide an intermediate of formula XI and subsequent condensation of XI with 2-[2-[3-(S)-[3-[2-(7-chloro-2-quinolinyl)-ethenyl]phenyl]-3-methanesulphonyloxypropyl]phenyl]-2-propanol at 0 to −50° C. to afford 1-[[[(1R)-1-[3-[(1E)-2-(7-chloro-2-quinolinyl)ethenyl]phenyl]-3-[2-(1-hydroxy-1-methylethyl)phenyl]propyl]thio]methyl]cyclopropane acetic acid as the quaternary ammonium salt which is filtered, purified by crystallization from a suitable solvent and finally treated with a sodium base in a suitable solvent to generate montelukast sodium which is isolated by trituration with an antisolvent. The invention provides a convenient process for preparing montelukast sodium (I) in good yields and purities.

### FIELD OF THE INVENTION

The present invention relates to an improved process for the manufacture of 1-[[[(1R)-1-[3-[(1E)-2-(7-chloro-2-quinolinyl)ethenyl]phenyl]-3-[2-(1-hydroxy-1-methylethyl)phenyl]propyl]thio]methyl]cyclopropane acetic acid, sodium salt I, which is known as Montelukast sodium.

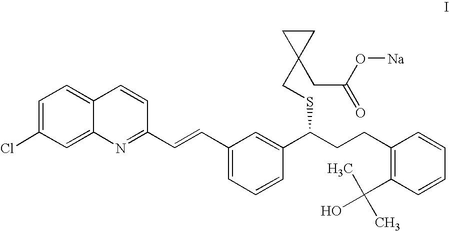

The compound of the formula I is a selective and orally active leukotriene receptor antagonist that inhibits the cysteinyl leukotriene CysLT₁ receptor. These compounds are effective in the treatment of asthmatic disorders, etc. Several processes for the manufacture of the same are reported.

### PRIOR ART

The European Patent No. 480717 discloses a class of novel anti-asthmatic compounds including montelukast sodium of structural formula I, having activity as leukotriene antagonists and to methods for their preparation. This patent provided a process for the preparation of the title compound I, which comprises of converting an alcohol of the formula II to a mesylate of the formula III. The mesylate is then condensed, in presence of cesium carbonate, with methyl (1-acetylthiomethyl)cyclopropaneacetate of the formula IV, after treatment of the latter with hydrazine, to obtain a compound of the formula V

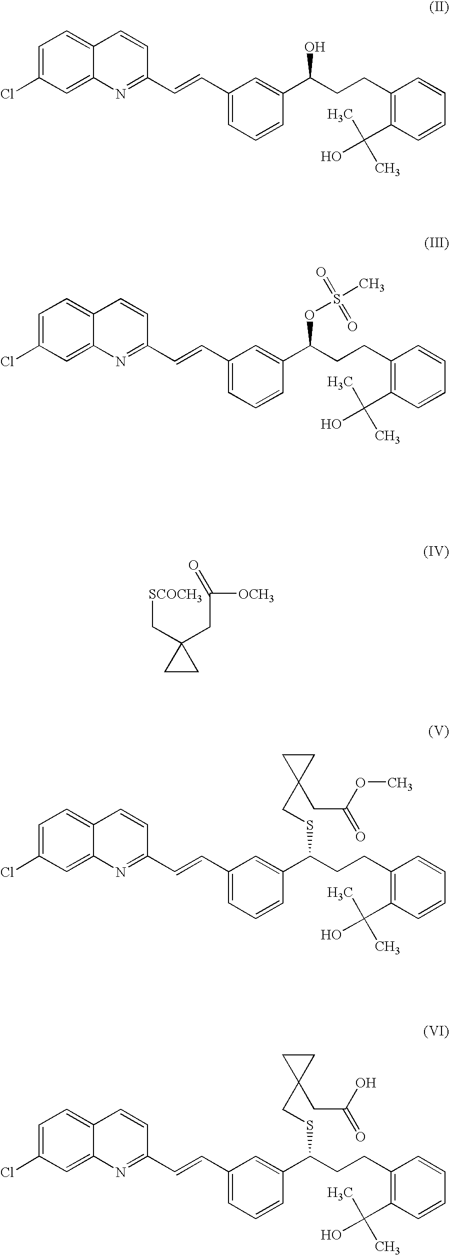

Finally the acid of the formula VI was prepared by hydrolysis of the methyl ester V in presence of pyridinium p-toluenesulphonate. The acid VI was, then, taken up in ethanol, treated with an equivalent amount of NaOH and the resultant oil was freeze dried to afford the compound of the formula I. This process afforded the title compound in low yields and purities and required purification by chromatography at intermediate stages.

European Patent No. 500360 relates to quinoline-containing ketoacids having activity as leukotriene antagonists and to methods for their preparation. This patent again provides processes as exemplified in EP 480717 and hence suffers from the same drawbacks.

The European Patent No. 737186 relates to a process for the preparation of a compound of the formula I which comprises of reacting the dilithium dianion of 1-(mercaptomethyl)cyclopropaneacetic acid (VII) with methanesulphonyloxy compound of the formula III to afford after suitable workup the acid VI which was in situ converted to its dicyclohexyl amine salt having the formula VIII.

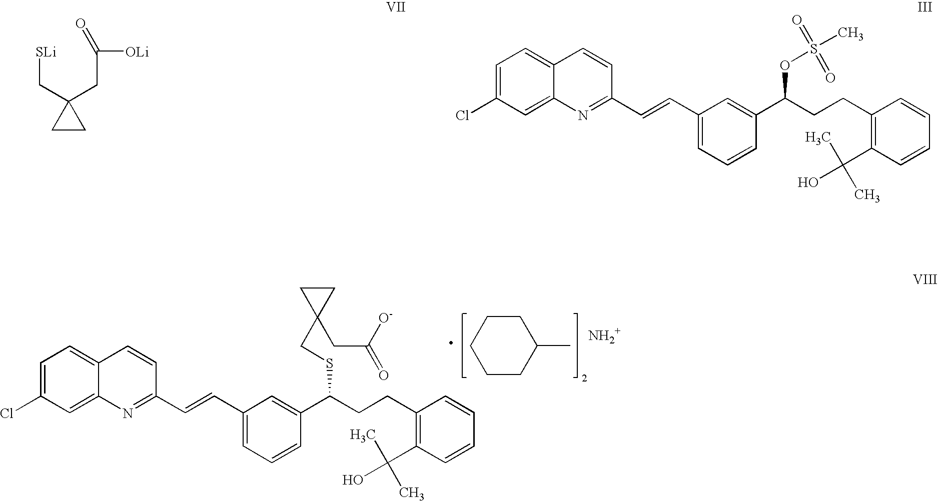

The dicyclohexylamine salt was purified by leaching with solvents and dried. The dried salt VIII was taken up in toluene and treated with acetic acid to generate free acid VI, the toluene solution of which was subsequently treated with an equivalent quantity of sodium hydroxide and the sodium salt so formed (I) was crystallized from a solvent mixture comprising of toluene-acetonitrile. This process suffers from multiplicity of steps involving formation of VI, conversion of the latter to its dicyclohexylamine salt, purification of the dicyclohexylamine salt, regeneration of acid VI before it is converted to montelukast sodium which is crystallized, making it very tedious and industrially unattractive.

The provisional patent application WO 03/066598 discloses an anhydrous amorphous form of montelukast sodium of the formula I which comprises of preparing the montelukast free acid from montelukast dicyclohexylamine salt by acidification, dissolving the free acid of montelukast in a C₁-C₂ halogenated solvent or in C₇-C₈ aromatic hydrocarbon solvent and converting the dissolved acid to the corresponding alkali salt using an alkaline metal hydroxide/an alkaline metal alkoxide/alcoholic alkaline metal hydroxide/alcoholic alkaline metal alkoxide in presence of C₁-C₄ straight or branched chain alcohol and isolating amorphous form of montelukast alkali salt by adding a C₅-C₇ acyclic or C₅-C₈ cyclic hydrocarbon. This process affords the compound of the formula in yields less than 70% of theory which renders the process unattractive.

The provisional patent application WO 04/108679 relates to an improved method for the preparation of montelukast acid sodium salt in an amorphous form which comprises of generating the dilithium dianion of I-(thiomethyl)cyclopropaneacetic acid (VII) and coupling said dianion with wet mesylate of the formula III to get montelukast acid VI in crude form followed by conversion of the latter to its DCHA salt, purifying the DCHA salt and converting the DCHA salt to montelukast acid in the pure form and finally reacting the pure montelukast acid with a sodium base followed by evaporation of the solvent and triturating the residue with nonpolar water immiscible solvent to obtain the title compound, I. This long drawn procedure affects the overall yield of the final product.

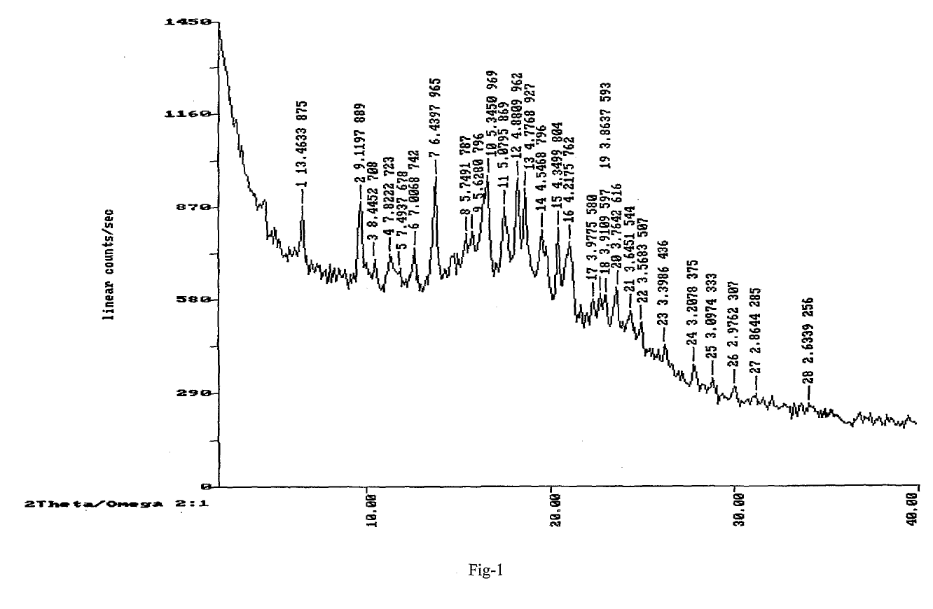

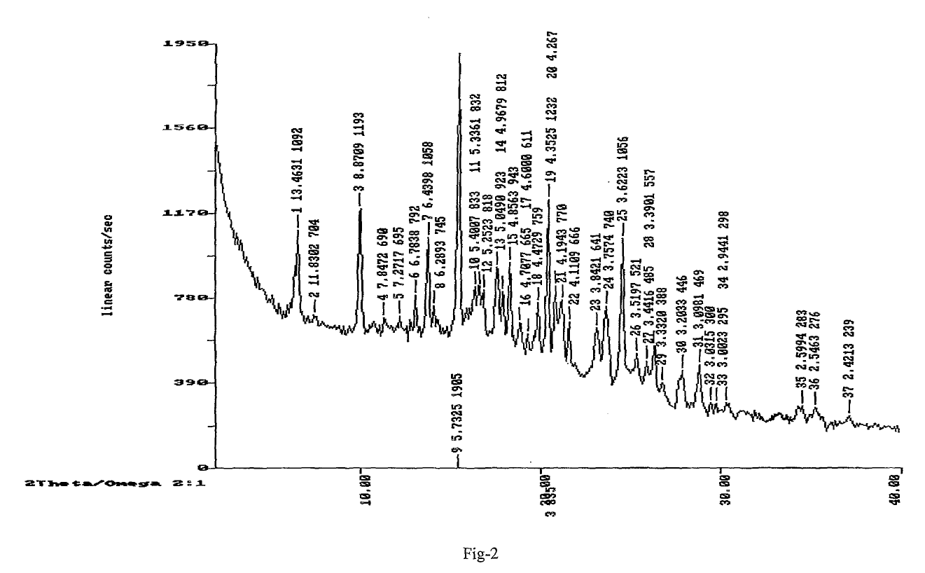

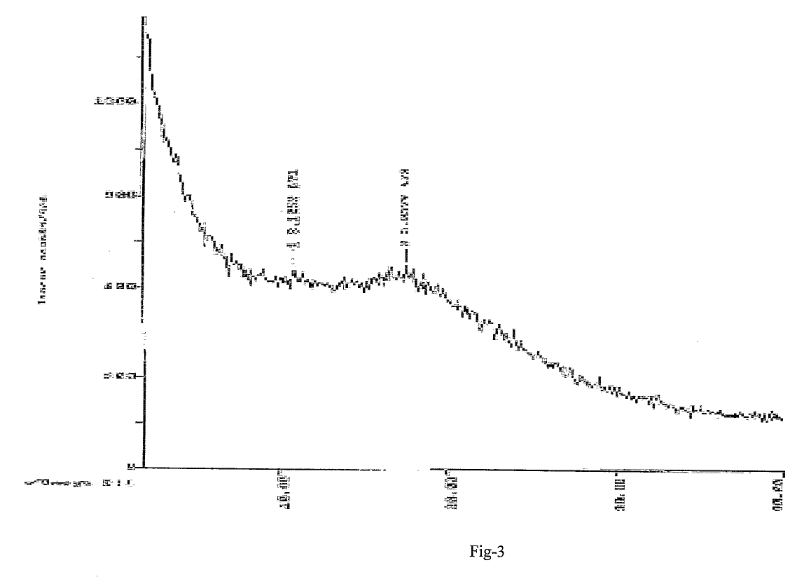

The US application US2005/0187245 discloses a stable non-hygroscopic amorphous form of the compound of the formula I which comprises of dissolving the montelukast sodium in a solvent/a combination of solvents followed by spray drying the resultant solution. As a comparative example the patent also reports that a product prepared according to the process disclosed in EP 480717 which comprises of freeze drying an aqueous solution of montelukast sodium, provides an amorphous form as confirmed by the X-ray Diffraction data of the product. We obtained similar results by repeating the process of freeze drying of montelukast as reported in EP 480717. The X-ray diffractogram of the product obtained is shown in FIG. 3. The patent, US2005/0187245, however does not disclose the yields obtained by following their procedure and is thus not clear.

### SUMMARY

The object of the present invention is to provide a process for the manufacture of the compound of formula I in good yields by reducing the number of steps while still achieving good purities.

Another objective of the present invention is to use a tertiary amine which serves a dual function of converting the carboxylic acid end of 1-(mercaptomethyl)-cyclopropane acetic acid, IX to its quaternary ammonium salt, X before condensation of monometal mercaptide derivative XI with mesylate III and causing the resulting quaternary ammonium salt, XII, of montelukast acid VI to directly crystallize out of the solvent media. Also the process of the present invention has lesser number of steps and it utilizes lesser quantity of alkyl metal.

Another object of the present invention is to provide a process for the manufacture of the compound of the formula I that is simple, easy and convenient to carry out.

Another object of the invention is to provide a process for the manufacture of the compound of the formula I that is economical and commercially viable.

### DETAILED DESCRIPTION

Accordingly, the present invention devises a more efficient process for montelukast sodium (I), it was conceived that carboxylic acid IX can be readily converted to quaternary ammonium salts (X) which can have potential uses for onward coupling with suitable substrates. The so-formed quaternary ammonium salts of the formula X apart from protecting the carboxylic acid during coupling, could also directly afford stable crystalline salts during suitable work-up procedures. The synthetic applicability of this basic concept to the objective of the present invention forms the basis of this invention.

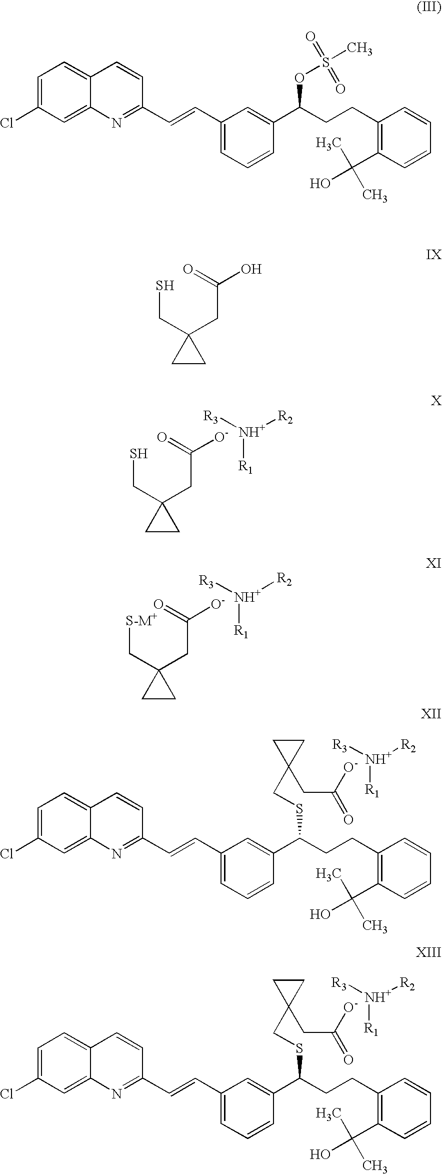

According to the invention there is provided a process for the manufacture of the compound of the formula I consisting of converting 1-(mercaptomethyl)-cyclopropane acetic acid of the formula IX to a quaternary ammonium salt of the formula X wherein R₁, R₂, R₃ can be independently alkyl, aralkyl, aryl or R₁, R₂, R₃, partially or wholly may form a cyclic substructure. This is then converted to a monometal salt of the formula XI wherein R₁, R₂, R₃ can be independently alkyl, aralkyl, aryl or R₁, R₂, R₃ partially or wholly may form a cyclic substructure and M⁺ can be from Li⁺, Na⁺, K⁺, Ca⁺², Mg⁺². This is subsequently condensed with the compound of the formula III in a suitable solvent at 0 to −50° C. followed by trituration of the reaction mass in a suitable solvent to afford a compound of the formula XII as a quaternary ammonium salt which is isolated by filtration. The compound of the formula XII is then purified by crystallization from a suitable solvent. It was important to establish that during the condensation of mesylate III with the monometal mercaptide XI, complete inversion occurs at the carbon carrying the mesyl group, to give the desired enantiomer XII and that the proportion of the undesired enantiomer XIII does not increase as compared to the standard procedure reported in EP 737186. This was done by preparing XII where the tertiary amine used is N-methylmorpholine and also preparing N-methylmorpholine salt of VI obtained according to the described in EP 737186. The two samples had identical specific optical rotation and X-ray diffraction pattern

To obtain montelukast sodium (I), the purified salt XII is dissolved in a suitable solvent and treated with a stoichiometric amount of a sodium base at 0 to 50° C. followed by trituration of the resultant solution in an antisolvent. An amorphous powder of pure montelukast sodium (I) is obtained.

As a suitable solvent for the quaternization of the compound of the formula IX affording X followed by monometalation affording XI, one can utilize the hydrocarbons such as hexane, n-heptane, cyclohexane, toluene, ethers such as diethyl ether, diisopropyl ether, methyl tertbutyl ether, THF, acetonitrile, preferably THF.

As a suitable solvent for the reaction between monometal salt of the formula XI and compound of the formula III, one can utilize the ethers such as dialkyl ethers, where alkyl connotes methyl, ethyl, n- & isopropyl, cyclic ethers such as THF, 1,4-dioxane, etc. More preferred ones are the cyclic ethers like tetrahydrofuran and 1,4-dioxane.

The molar quantity of the base used for quaternization can be varied between 1.0 and 1.5 but preferably 1.1-1.2 moles with respect to the compound of the formula IX.

The base used for quaternization has the general formula NR₁R₂R₃, wherein R₁, R₂, R₃ can be independently alkyl, aralkyl, aryl or R₁, R₂, R₃, partially or wholly may form a cyclic substructure and is selected from a group comprising of the trialkylamines such as triethylamine, trimethylamine, tri n- and iso-propylamines, tributylamine, N-methylmorpholine, pyridine, lutidines, picolines etc, preferably N-methylmorpholine.

The molar quantity of monometal mercaptide XI used with respect to the compound of the formula III can be varied between 1.0-2.0 moles but preferably 1.5 to 1.6 moles with respect to the compound of the formula III.

The condensation reaction between the monometal mercaptide XI and the compound of the formula III is carried out between 0 to −50° C. preferably −25° C.

As an organic solvent for dissolving the reaction mass residue one can utilize hydrocarbons such as hexane, n-heptane, cyclohexane, toluene, ethers such as diethyl ether, diisopropyl ether, methyl tertbutyl ether, THF, acetonitrile, preferably toluene.

As an organic solvent for trituration in order to isolate the compound of the formula XII one can utilize suitable antisolvents for precipitating the compound of the formula XII which are selected from hydrocarbons such as hexane, n-heptane, cyclohexane, toluene, ethers such as diethyl ether, diisopropyl ether, methyl tertbutyl ether, preferably hexane.

As an organic solvent for purification of the quaternary ammonium salt of the formula XII one can utilize halogenated organic solvents, ethers, alkyl acetates, aromatic hydrocarbons, etc. More preferred are the alkyl acetates preferably ethyl acetate.

The organic solvent for dissolving the purified compound of the formula XII is selected from hydrocarbons such as hexane, n-heptane, cyclohexane, toluene, ethers such as diethyl ether, diisopropyl ether, methyl tertbutyl ether, ketones such as acetone, methyl ethyl ketone or methyl isobutyl ketone or esters such as methyl acetate, ethyl acetate or n-butyl acetate. More preferred is toluene.

As a base for generating the sodium salt one can utilize the alkali metal hydroxides such as sodium hydroxide, the alkali metal carbonates such as sodium carbonate, alkali metal acetates such as sodium acetate, or alkali metal alkoxides such as sodium methoxide. More preferred is sodium methoxide.

The suitable antisolvent for precipitating the compound of the formula I is selected from hydrocarbons such as hexane, n-heptane, cyclohexane, toluene, ethers such as diethyl ether, diisopropyl ether, methyl tertbutyl ether, ketones such as acetone, methyl ethyl ketone or methyl isobutyl ketone or esters such as methyl acetate, ethyl acetate or n-butyl acetate, preferably n-heptane.

The process does not proceed via the dilithio salt. The process of the present invention does not employ the dicyclohexylamine salt as an intermediate. The process of the present invention does not utilize any freeze dryer for the isolation of the compound of the formula I. The process of the present invention does not proceed via the montelukast free acid.

The process of the invention does not use organic bases like dicyclohexyl amine for isolation. The process of the invention does not utilize solvents such as acetonitrile during the final stages of crystallization, which have a stringent limit in ICH.

The following examples are illustrative of the invention but not limitative to the scope thereof.

### Example 1

### Montelukast N-methyl Morpholine Salt

A suspension of 1-(mercaptomethyl)-cyclopropane acetic acid (5.0 gm, 0.034 mol) (IX) and N-metlhylmorpholine (3.74 gm, 0.037 mol) in 40 ml THF was stirred at 25° C. for 2 hrs and cooled to −40° C. To this 23 ml of n-butyl lithium (0.037 mol) was added followed by maintaining the reaction mass at −20 to −40° C. for an additional 30 min. Mesyl derivative (III) (12 gm, 0.022 mol) dissolved in 20 ml THF was added to the reaction mass and the reaction was stirred at −5 to −10° C. for 12 hrs. The reaction mass was concentrated in vacuum and dissolved in 50 ml toluene. The solution was treated with charcoal, filtered through celite and triturated with 300 ml hexane. The resultant salt was filtered and purified by crystallization from 20 ml ethyl acetate to afford 12.8 gm of the N-methyl morpholine salt of montelukast. Water content (by Karl Fisher)=0.2%; Chemical assay (by HPLC) 98.7%; Yield=83.3% (of theory)

I.R (cm⁻¹): 3409, 1712, 1604, 1496
¹H NMR (CDCl3): δ 8.10-8.14 (d, 1H), 8.05-8.06 (d, 1H), 7.67-7.76 (m, 3H), 7.61 (bs, 1H), 7.26-7.50 (m, 6H), 7.08-7.15 (m, 3H), 4.01-4.05 (t, 1H), 3.11-3.36 (m, 8H), 2.72-2.95 (m, 2H), 2.22-2.65 (m, 4H), 2.04-2.18 (m, 2H), 1.95 (d, 1H), 1.75 (m, 1H), 1.60-1.62 (2s, 6H), 1.17-1.29 (m, 2H), 0.45-0.53 (m, 4H)

### SOR: +78.94

### XRD: As per FIG.-1

### Montelukast Sodium Salt

The above N-methyl morpholine salt (10 gm, 0.014) was dissolved in 80 ml toluene and to the resultant solution 0.86 μm of sodium methoxide (0.016 mol) was added and the contents stirred for 30 min at 25-30° C. followed by addition of 0.5 gm charcoal. The mass was stirred at 25-30° C. for 1 hr and filtered through celite. The clear filtrate was added drop-wise into 300 ml n-heptane. The product mass was stirred for an additional 10 min at 25-30° C. and filtered. The product was dried at 50° C. under vacuum to get 7.95 gm of montelukast sodium. Water content (by Karl Fisher)=1.8%; Chemical assay (by HPLC) 99.2%; Yield 90% (of theory); SOR: +79.12

### Example 2

The procedure of example 1 was followed with 4.03 gm of 2,6-lutidine instead of N-methylmorpholine to afford the 2,6-lutidine salt of montelukast.

I.R (cm⁻¹): 3417, 1712, 1604, 1498
¹H NMR (CDCl₃): δ 8.10-8.14 (d, 1H), 8.05-8.06 (d, 1H), 7.68-7.78 (m, 3H), 7.60 (bs, 1H), 7.26-7.52 (m, 9H), 7.08-7.15 (m, 3H), 4.02-4.05 (t, 1H), 2.88-3.15 (m, 8H), 2.42-2.74 (m, 2H), 2.18-2.42 (m, 3H), 2.04 (m, 1H), 1.61-1.63 (2s, 6H), 1.17-1.26 (m, 1H), 0.47-0.54 (m, 4H)

### SOR: +79.12

XRD: As per FIG.-2

1. A process for the preparation of 1-[[[(1R)-1-[3-[(1E)-2-(7-chloro-2-quinolinyl)ethenyl]phenyl]-3-[2-(1-hydroxy-1-methylethyl)phenyl]propyl]thio]methyl]cyclopropane acetic acid, sodium salt [montelukast sodium (I)], said process comprising steps of:
(a) treating 1-(mercaptomethyl)-cyclopropane acetic acid of formula IX, in 6-10 volumes of a suitable solvent under inert atmosphere, such as provided by nitrogen, helium or argon, with one mole equivalent of a tertiary amine at a temperature in the range of 0 to 50° C. affording the quaternary salt of the structure X;
(b) treating the compound of the formula X with one mole equivalent of a metalide forming substance to obtain trialkylammonium 1-metalomercapto-1-cyclopropane acetate of the formula XI, at a temperature ranging from −50 to 0° C.;
(c) mixing a solution of 2-(2-(3-(S)-(3-(2-(7-chloro-2-quinolinyl)-ethenyl)phenyl-3-methanesulphonyloxypropyl)phenyl)-2-propanol (III) in 1.0 to 2.0 volumes of a suitable solvent at a temperature in the range of −20 to 0° C. with a solution of 1.1 to 1.5 moles of XI cooled to −20 to 0° C. maintained at that temperature for 8-20 hrs and the solvent was removed under reduced pressure;
(d) dissolving the residue in 5-8 volumes of a suitable solvent and pouring the solution into 30-40 volumes of an antisolvent at temperatures ranging from 0-50° C. to let XII crystallize out;
(e) purifying XII by crystallization from 3 to 5 volumes of a suitable solvent at 50-80° C. and cooling to ambient temperature;
(f) dissolving purified XII in 8-10 volumes of a suitable solvent and treating it with an equivalent quantity of sodium base at 25 to 50° C.; and
(g) adding 25-50 volumes of an antisolvent and keeping the contents at 25 to 50° C. to obtain precipitate of compound of formula I.

2. The process as claimed in claim 1, wherein in step (a) the suitable solvent for converting the compound of the formula IX to a quaternary ammonium salt of formula X is selected from hydrocarbons comprising hexane, n-heptane, cyclohexane, toluene, ethers such as diethyl ether, diisopropyl ether, methyl tertbutyl ether, THF, and acetonitrile.

3. The process as claimed in claim 1, wherein in step (a) the tertiary amine used for converting the compound IX to the quaternary ammonium salt has the general formula NR₁R₂R₃, wherein R₁, R₂, R₃ can be independently alkyl, aralkyl, aryl or R₁, R₂, R₃, partially or wholly may form a cyclic substructure and is selected from a group comprising of the trialkylamines such as triethylamine, trimethylamine, tri n- and iso-propylamines, tributylamine, N-methylmorpholine, pyridine, lutidines, and picolines.

4. The process as claimed in claim 1, wherein in step (b) the suitable metalide forming substance for converting the compound of the formula X to a compound of the formula XI is selected from lithium forming substances such as n-, sec- & tert-butyl lithium, lithium hydride, sodium forming substance such as sodium hydride, sodium methoxide, sodium hydroxide, phenyl sodium, potassium forming substance such as potassium hydride, potassium methoxide, potassium hydroxide, calcium forming substance such as calcium hydride, calcium hydroxide, magnesium forming substance such as magnesium oxide, and magnesium hydroxide.

5. The process as claimed in claim 1, wherein in step (c) the suitable solvent for dissolving the compound of the formula III is selected from hydrocarbons such as hexane, n-heptane, cyclohexane, toluene, ethers such as diethyl ether, diisopropyl ether, methyl tertbutyl ether, THF, and acetonitrile.

6. The process as claimed in claim 1, wherein in step (d) the suitable solvent for dissolving the reaction mass residue is selected from hydrocarbons such as hexane, n-heptane, cyclohexane, toluene, ethers such as diethyl ether, diisopropyl ether, methyl tertbutyl ether, THF, and acetonitrile.

7. The process as claimed in claim 1, wherein in step (d) the suitable antisolvent for precipitating the compound of the formula XII is selected from hydrocarbons such as hexane, n-heptane, cyclohexane, toluene, ethers such as diethyl ether, diisopropyl ether, and methyl tertbutyl ether.

8. The process as claimed in claim 1, wherein in step (e) the suitable solvent for purifying the compound of the formula XII is selected from hydrocarbons such as hexane, n-heptane, cyclohexane, toluene, ethers such as diethyl ether, diisopropyl ether, methyl tertbutyl ether, ketones such as acetone, methyl ethyl ketone or methyl isobutyl ketone, and esters such as methyl acetate, ethyl acetate or n-butyl acetate.

9. The process as claimed in claim 1, wherein in step (f) the suitable solvent for dissolving the purified compound of the formula XIII is selected from hydrocarbons such as hexane, n-heptane, cyclohexane, toluene, ethers such as diethyl ether, diisopropyl ether, methyl tertbutyl ether, ketones such as acetone, methyl ethyl ketone or methyl isobutyl ketone, and esters such as methyl acetate, ethyl acetate or n-butyl acetate.

10. The process as claimed in claim 1, wherein in step (f) the suitable base for converting the purified compound of the formula XII to the sodium salt is selected from sodium hydroxide, sodium carbonate, sodium bicarbonate, and sodium alkoxide, where alkoxide connotes methoxide, ethoxide, n- & iso-propoxide.

11. The process as claimed in claim 1, wherein in step (g) the suitable antisolvent for precipitating the compound of the formula I is selected from hydrocarbons such as hexane, n-heptane, cyclohexane, toluene, ethers such as diethyl ether, diisopropyl ether, methyl tertbutyl ether, ketones such as acetone, methyl ethyl ketone or methyl isobutyl ketone, and esters such as methyl acetate, ethyl acetate or n-butyl acetate.

12. (canceled)
